(19) United States
(12) Patent Application Publication
(10) Pub. No.: US 2023/0053539 A1
(43) Pub. Date: Feb. 23, 2023

(54) NURSE CALL SYSTEM, METHOD FOR NURSE CALL AND PROGRAM

(71) Applicant: NEC Platforms, Ltd., Kanagawa-shi, Kanagawa (JP)

(72) Inventor: Hatsue NIIYA, Kanagawa (JP)

(73) Assignee: NEC Platforms, Ltd., Kanagawa-shi, Kanagawa (JP)

(21) Appl. No.: 17/795,673

(22) PCT Filed: Jan. 20, 2021
(86) PCT No.: PCT/JP2021/001752

§ 371 Date: Jul. 27, 2022

(30) Foreign Application Priority Data
Feb. 10, 2020 (JP) 2020-020462

(51) Int. Cl. G16H 40/20; A61G 12/00

(52) U.S. Cl. CPC G16H 40/20; A61G 12/00

(57) ABSTRACT

A nurse call system of the present disclosure includes: a nurse call slave unit; a nurse call master unit; a mobile terminal; an access point for relaying communications between the mobile terminal and the nurse call master unit; a private branch exchange configured to control communications between the nurse call slave unit and the mobile terminal; and a chat server configured to generate a chat room to be delivered to the mobile terminal in response to a call originated from the nurse call slave unit, in which the chat server generates a chat room with in-charge healthcare workers in charge of a subject patient who originated a call using the nurse call slave unit and a nearby healthcare worker assumed to be at a location closer to the subject patient than the in-charge healthcare workers are as users, and provides the chat room to each mobile terminal.

### TECHNICAL FIELD

The present disclosure relates to a nurse call system, in particular, to a nurse call system for supporting a plurality of nurses working together to take care of a patient.

### BACKGROUND ART

In recent years, team nursing in which a plurality of healthcare workers work together to provide nursing care for one patient has been widely adopted. A nurse call system for supporting such team nursing has been proposed. An example of such nurse call system is disclosed in Patent Literature 1.

A nurse call system described in Patent Literature 1 includes:
a nurse call slave unit;
a nurse call master unit;
a mobile terminal carried by a healthcare worker;
a nurse call controller configured to control communications performed among the nurse call slave unit, the nurse call master unit, and the mobile terminal;
a private branch exchange for controlling calls between the nurse call slave unit, the nurse call master unit, and the mobile terminal; and
a generation unit connected to the nurse call controller and the mobile terminal in a communicable manner and configured to generate a chat room where a message can be posted in a chat style based on operation of the mobile terminal by the healthcare worker,
in which the chat room is displayable on each mobile terminal carried by a plurality of healthcare workers belonging to a team in charge of a given patient.

### CITATION LIST

### Patent Literature

Patent Literature 1: Japanese Unexamined Patent Application Publication No. 2019-140428

### SUMMARY OF INVENTION

### Technical Problem

However, in the nurse call system described in Patent Literature 1, the chat room is created only for in-charge nurses who are pre-set to be in charge of the care of the patient. Therefore, even when the in-charge nurses are at locations distant from the location where the patient is at while a nurse who is not the in-charge nurse is at a location close to the location where the patient is at, the nurse who is not in charge of the patient but is at a location close to the location where the patient is at is not invited to the chat room while the in-charge nurses, who are at locations distant from the location where the patient is at, need to respond to the invitation. In other words, with the nurse call system described in Patent Literature 1, work efficiency of team nursing cannot be sufficiently improved.

### Solution to Problem

A nurse call system according to an example embodiment includes:
a nurse call slave unit provided for patient-use;
a nurse call master unit having the slave unit connected thereto;
a mobile terminal carried by a healthcare worker;
an access point for relaying communications between the mobile terminal and the nurse call main unit;
a private branch exchange having a plurality of the nurse call master units installed in a facility connected thereto and being configured to control communications between the nurse call slave unit and the mobile terminal; and
a chat server configured to generate a chat room to be delivered to the mobile terminal in response to a call originated from the nurse call slave unit, in which
the private branch exchange has first information indicating corresponding relationship between the mobile terminal and the healthcare worker, second information indicating corresponding relationship between the patient and an in-charge healthcare worker who is in charge of the patient among the healthcare workers, and third information indicating location information of the nurse call slave unit and the mobile terminal, and
the chat server generates, based on the first and the third information, a chat room with the in-charge healthcare worker in charge of a subject patient who originated a call using the nurse call slave unit and a nearby healthcare worker assumed to be at a location closer to the subject patient than the in-charge healthcare worker is as users, and provides the chat room to each mobile terminal held by the in-charge healthcare worker and the nearby healthcare worker.

### Advantageous Effects of Invention

Work efficiency of team nursing can be improved by a nurse call system according to an example embodiment.

### BRIEF DESCRIPTION OF DRAWINGS

FIG. 1 is a block diagram of a nurse call system according to a first example embodiment; and

FIG. 2 is a flowchart for describing operation of the nurse call system according to the first example embodiment.

### EXAMPLE EMBODIMENT

### First Example Embodiment

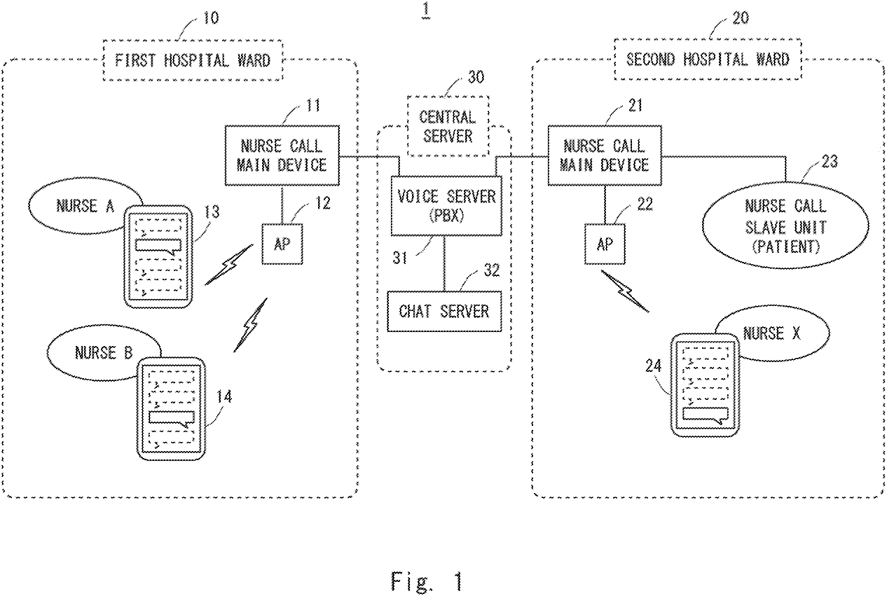

Hereinbelow, an example embodiment of the present disclosure will be described with reference to the drawings. FIG. 1 shows a block diagram of a nurse call system 1 according to the first example embodiment. In the example shown in FIG. 1, the nurse call system 1 is utilized in a facility consisting of two separate buildings, a first hospital ward 10 and a second hospital ward 20. Note that in the actual nurse call system 1, nurse call master units (e.g. nurse call main devices), nurse call slave units, and mobile terminals may be employed by a number greater than those in the example shown in FIG. 1. Further, in the example shown in FIG. 1, a nurse call main device is installed for each hospital ward, but the system may be configured with one nurse call main device installed for shared-use by multiple hospital wards or multiple floors.

In the example shown in FIG. 1, a central server 30 is employed for equipment of shared-use by the first hospital ward 10 and the second hospital ward 20. The central server 30 includes a voice server 31 and a chat server 32. Further, the first hospital ward 10 is equipped with a nurse call main device 11, an access point 12, a mobile terminal 13, and a mobile terminal 14. The second hospital ward 20 is equipped with a nurse call main device 21, an access point 22, a nurse call slave unit 23, and a mobile terminal 24.

The nurse call slave unit 23 is provided for patient-use. To be more specific, the nurse call slave unit 23 is installed so as to be attached to the bed used by the patient. That is, the nurse call slave unit 23 is installed at a fixed location. Further, the location information of the nurse call slave unit 23 is registered in the nurse call main device and the private branch exchange at the time of installation of the nurse call slave unit 23. The nurse call slave unit 23 originates a nurse call, for instance, when a patient presses a button. The nurse call main devices 11 and 21 have the nurse call slave unit 23 and the access points 12 and 22 connected thereto. In the example shown in FIG. 1, the nurse call main device 11 has the access point 22 connected thereto, and the nurse call main device 21 has the nurse call slave unit 23 and the nurse call main device 21 connected thereto.

The mobile terminals 13 and 14 and the potable terminal 24 are terminals carried by the healthcare workers. As the mobile terminal, a terminal capable of running a software such as an on-premises PHS (personal handy-phone system), a mobile information terminal, a tablet terminal, and the like can be utilized. The access points 12 and 22 relay communication between the mobile terminals and the aforementioned nurse call main devices. The access points 12 and 22 function as a master unit of the wireless LAN when the terminals that belong to the access points are terminals such as a mobile phone or the like. The access points 12 and 22 function as a radio base station when the terminals that belong to the access points are PHSs or the like. Further, the mobile terminals are connected to the nurse call main device via the access points. In the example shown in FIG. 1, the mobile terminals 13 and 14 are connected to the nurse call main device 11 via the access point 12. Further, the mobile terminal 24 is connected to the nurse call main device 21 via the access point 22. Here, the access points and the mobile terminals perform communication utilizing a radio signal. Note that in the example shown in FIG. 1, the mobile terminal 13 is allocated to Nurse A, the mobile terminal 14 is allocated to Nurse B, and the mobile terminal 24 is allocated to Nurse X. Further, in the nurse call system 1 according to the first example embodiment, it is assumed that the corresponding relationship between the nurses and the mobile terminals allocated to the nurses is held in the voice server 31.

The voice server 31 is, for example, a PBX (Private Branch eXchange). The voice server 31 has the nurse call main devices 11 and 21 connected thereto, the nurse call main devices being installed within the facility by a plural number, and is configured to control communication between the nurse call slave unit 23 and the mobile terminals 13, 14, and 24. In response to a call originated from the nurse call slave unit 23, the chat server 32 generates a chat room to be delivered to the mobile terminals 13, 14, and 24.

Here, in the nurse call system 1 according to the first example embodiment, the voice server 31 has first to third information. The first information is information indicating corresponding relationship between the mobile terminals 13, 14, and 24 and healthcare workers (e.g. Nurse A, Nurse B, and Nurse X). The second information is information indicating corresponding relationship between the patient and in-charge healthcare workers who are in charge of the patient among the nurses (e.g. in-charge nurses). The third information is information indicating location information of the nurse call slave unit 23 and the mobile terminals 13, 14, and 24.

Since the nurse call slave unit 23 is installed at a fixed location, the location information of the nurse call slave unit 23 is registered as the third information at the time of installation of the nurse call slave unit 23. Further, the location information of the mobile terminals includes at least one of the location information of the mobile terminals acquired using a location information acquisition function (e.g. the GPS (Global Positioning System)) built-in in the mobile terminals and the location information of the access points to which the mobile terminals belong. Note that since the access points are installed at fixed locations, the location information of the access points is registered as the third information at the time of installation of the access points.

Further, the chat server 32 generates, based on the first to the third information, a chat room with the in-charge nurses who are in charge of a subject patient who originated a call using the nurse call slave unit 23 and a nearby healthcare worker assumed to be at a location closer to the subject patient than the in-charge nurses are (e.g. a nearby nurse) as users, and provides the chat room to the mobile terminals 13, 14, and 24 held by the in-charge nurses and the nearby nurse. Here, when the location relationship between the mobile terminals and the nurse call slave unit is determined based on the location information of the access points, the chat server 32 simply uses the locations of the access points to which the mobile terminals belong as the locations of the mobile terminals.

Note that in the example shown in FIG. 1, Nurse A and Nurse B are the in-charge nurses who have corresponding relationship with the nurse call slave unit 23, and Nurse X is the nearby nurse. Further, FIG. 1 shows a state in which at the timing when a call is originated from the nurse call slave unit 23, the in-charge Nurses A and B are at a first hospital ward 10 that is different from a second hospital ward 20 where the patient is at, and in the case where the in-charge Nurses A and B cannot take prompt action in response to the call, the nearby Nurse X is requested to take care of the patient.

In the nurse call system 1 according to the first example embodiment, the access point 12 generates a chat room that includes as users the nearby Nurse X in addition to the in-charge Nurses A and B. By this configuration, in the nurse call system 1 according to the first example embodiment, improvement in the operating efficiency of team nursing is realized. Next, operation of the nurse call system 1 according to the first example embodiment will be described hereinbelow.

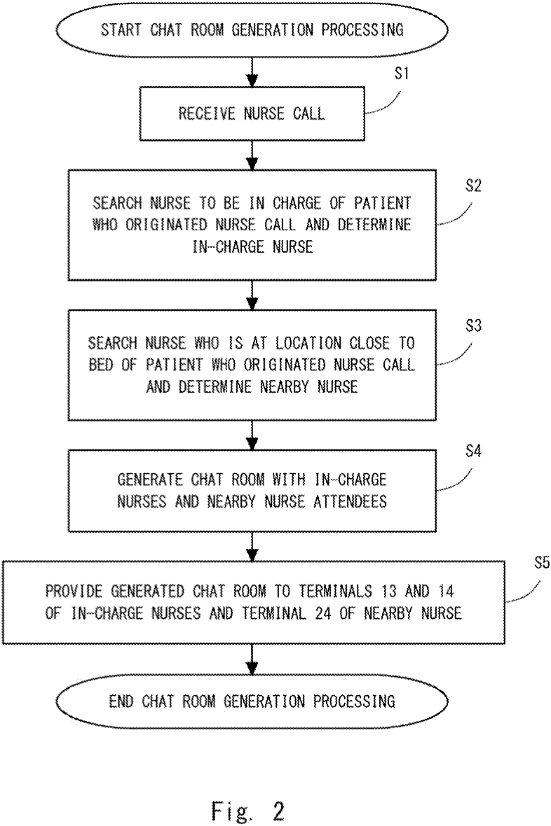

FIG. 2 shows a flowchart describing operation of the nurse call system 1 according to the first example embodiment. As shown in FIG. 2, in the nurse call system 1 according to the first example embodiment, chat room generation processing is started when a call is originated from the nurse call slave unit 23 (Step S1). Then, in the nurse call system 1 according to the first example embodiment, the chat server 32 receives the first to the third information from the voice server 31 and performs processing of Steps S1 to S5. In Step S2, a nurse to be in charge of the patient who originated the nurse call (e.g. the subject patient) is searched from the first and the second information and the in-charge nurse is determined. In Step S3, a nurse who is at a location close to the bed of the subject patient who originated the nurse call is searched and determined as the nearby nurse.

In the nurse call system 1 according to the first example embodiment, the access points are installed at fixed locations, and the voice server 31 generates the third information by estimating each mobile terminal from the location of the access point with which each mobile terminal is performing communication. Further, in the nurse call system 1 according to the first example embodiment, the mobile terminal switch the access point with which it performs communication as the nurse moves. Then, the voice server 31 updates the third information every time the access point with which the mobile terminal performs communication is switched. Further, the third information includes location information of the nurse call slave unit. Further, in the exploring processing of Step S3, based on the aforementioned third information, the nearby nurse who is at a location close to the patient is determined in accordance with the location of the nurse call slave unit from which the nurse call was originated and the mobile terminal which is at a location close to the aforementioned nurse call slave unit. Note that the nearby nurse is a nurse who is at a location closer to the subject patient than the in-charge nurses are. Alternatively, the nearby nurse may select a nurse who is close to the subject patient locationwise.

Next, in Step S4, a chat room with the in-charge nurses and the nearby nurse as attendees is generated. Then, in Step S5, the chat room generated in Step S4 is provided to the mobile terminals 13 and 14 held by the in-charge nurses (e.g. nurses A and B in FIG. 1) and the mobile terminal 24 held by the nearby nurse (e.g. nurse X in FIG. 1).

As described above, in the nurse call system 1 according to the first example embodiment, even when the in-charge nurses are at locations distant from the location where the subject patient is at, they can request, through the chat room, the nearby nurse who is nearby the subject patient to provide care for the subject patient. By this configuration, it is possible to improve the efficiency of team nursing in the nurse call system 1 according to the first example embodiment.

The present disclosure has been described with reference to the example embodiments. However, it should be noted that the present disclosure is to be limited in any way by the example embodiments described above. The configuration and details of the present disclosure can be modified in various ways within the scope of the disclosure which can be understood by a skilled person in the art.

This application is based upon and claims the benefit of priority from Japanese patent application No. 2020-20462, filed on Feb. 10, 2020, the entire contents of which are incorporated herein by reference.

### REFERENCE SIGNS LIST

1 NURSE CALL SYSTEM
10 FIRST HOSPITAL WARD
11 NURSE CALL MAIN DEVICE
12 ACCESS POINT
13 MOBILE TERMINAL
14 MOBILE TERMINAL
20 SECOND HOSPITAL WARD
21 NURSE CALL MAIN DEVICE
22 ACCESS POINT
23 NURSE CALL SLAVE UNIT
24 MOBILE TERMINAL
30 CENTRAL SERVER
31 VOICE SERVER
32 CHAT SERVER

What is claimed is:

1. A nurse call system comprising:
a nurse call slave unit provided for patient-use;
a nurse call master unit having the nurse call slave unit connected thereto;
a mobile terminal carried by a healthcare worker;
an access point for relaying communications between the mobile terminal and the nurse call master unit;
a private branch exchange having a plurality of the nurse call master units installed in a facility connected thereto and being configured to control communications between the nurse call slave unit and the mobile terminal; and
a chat server configured to generate a chat room to be delivered to the mobile terminal in response to a call originated from the nurse call slave unit, wherein
the private branch exchange has first information indicating corresponding relationship between the mobile terminal and the healthcare worker, second information indicating corresponding relationship between the patient and an in-charge healthcare worker who is in charge of the patient among the healthcare workers, and third information indicating location information of the nurse call slave unit and the mobile terminal, and
the chat server generates, based on the first and the third information, a chat room with the in-charge healthcare worker in charge of a subject patient who originated a call using the nurse call slave unit and a nearby healthcare worker assumed to be at a location closer to the subject patient than the in-charge healthcare worker is as users, and provides the chat room to each mobile terminal held by the in-charge healthcare worker and the nearby healthcare worker.

2. The nurse call system according to claim 1, wherein the private branch exchange updates the third information based on at least one of location information of the access point with which the mobile terminal performs communication and location information acquired by the mobile terminal.

3. The nurse call system according to claim 2, wherein when location relationship between the mobile terminal and the nurse call slave unit is determined based on the location information of the access point, the chat server simply uses the location information of the access point to which the mobile terminal belongs as the location of the mobile terminal.

4. The nurse call system according to claim 2, wherein the mobile terminal acquires the location information based on the Global Positioning System and transmits the location information acquired in response to a request from the private branch exchange to the private branch exchange.

5. The nurse call system according to claim 1, wherein the chat server and the private branch exchange are built-in in one device.

6. The nurse call system according to claim 1, wherein the access point is provided for each facility where the health care workers work, each floor of the facility, or each work area on the same floor of the facility.

7. The nurse call system according to claim 1, wherein the chat server selects a healthcare worker who is at a location closest to the subject patient as the nearby healthcare worker.

8. The nurse call system according to claim 2, wherein the chat server selects a healthcare worker who is at a location closest to the subject patient as the nearby healthcare worker.

9. The nurse call system according to claim 3, wherein the chat server selects a healthcare worker who is at a location closest to the subject patient as the nearby healthcare worker.

10. The nurse call system according to claim 4, wherein the chat server selects a healthcare worker who is at a location closest to the subject patient as the nearby healthcare worker.

11. The nurse call system according to claim 5, wherein the chat server selects a healthcare worker who is at a location closest to the subject patient as the nearby healthcare worker.

12. The nurse call system according to claim 6, wherein the chat server selects a healthcare worker who is at a location closest to the subject patient as the nearby healthcare worker.

13. A nurse call method for a nurse call system including a nurse call slave unit provided for patient-use and a mobile terminal carried by a healthcare worker, the method comprising:
generating, based on first to third information, a chat room with an in-charge healthcare worker in charge of a subject patient who originated a call using the nurse call slave unit and a nearby healthcare worker assumed to be at a location closer to the subject patient than the in-charge healthcare worker is as users,
the first information indicating corresponding relationship between the mobile terminal and the healthcare worker,
the second information indicating corresponding relationship between the patient and an in-charge healthcare worker who is in charge of the patient among the healthcare workers, and
the third information indicating location information of the nurse call slave unit and the mobile terminal; and
providing the chat room to each mobile terminal held by the in-charge healthcare worker and the nearby healthcare worker.

14. The nurse call method according to claim 13, wherein a healthcare worker who is at a location closest to the subject patient is selected as the nearby healthcare worker.

15. A non-transitory computer-readable medium storing a program for causing a computer to execute the nurse call method according to claim 13.

16. A non-transitory computer-readable medium storing a program for causing a computer to execute the nurse call method according to claim 14.
